(19) United States
(12) Patent Application Publication
(10) Pub. No.: US 2003/0087254 A1
(43) Pub. Date: May 8, 2003

(54) Methods for the preparation of polynucleotide libraries and identification of library members having desired characteristics

(76) Inventor: Simon Delagrave, Avondale, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/114,379

(22) Filed: Apr. 2, 2002

Related U.S. Application Data
(60) Provisional application No. 60/281,587, filed on Apr. 5, 2001.

(51) Int. Cl. C12Q 1/68; C07H 21/04; C12P 19/34

(52) U.S. Cl. 435/6; 435/91.2; 536/23.1

(57) ABSTRACT

Methods of directed fragmentation of polynucleotides combined with fragment interchange and ligation are provided for the preparation of polynucleotide libraries. Fragmentation can be facilitated by at least one oligonucleotide adapter capable of directing polynucleotide cleavage at homologous sites among a set of parent polynucleotides. Libraries generated by the above methods can be screened for polynucleotides with desired characteristics or properties.

### CROSS REFERENCE TO RELATED APPLICATIONS

This application claims the benefit of U.S. Provisional Application No. 60/281,587, filed Apr. 5, 2001, which is incorporated herein by reference in its entirety.

### FIELD OF THE INVENTION

The present invention relates to methods for the preparation of polynucleotide libraries and the identification of polynucleotides therefrom having desired properties.

### BACKGROUND OF THE INVENTION

Recombination of polynucleotides can be carried out by many methods known in the art. One such method includes DNA shuffling, which is described in Stemmer, et al., Proc. Natl. Acad. Sci. USA, 1994, 91, 10747; and U.S. Pat. Nos. 6,117,679, 6,165,793, and 6,153,410. Generally, DNA shuffling involves the fragmentation of several homologous genes and reassembly of the fragments to generate a large number of different polynucleotides.

While demostratably an efficient method for generating large DNA libraries from genes, DNA shuffling can have several disadvantages. For example, assembly of recombined polynucleotides proceeds via hybridization of complementary or partially complementary polynucleotide fragments. This requirement for hybridization limits the shuffling method to polynucleotides with a certain minimal amount of homology (>70% or sometimes >90%). Moreover, recombination between polynucleotides tends to occur at points of high sequence identity that are found randomly along the sequences. There is, therefore, little control of the sites of recombination during a shuffling experiment. Additionally, once the fragments are hybridized to each other, they are assembled into full-length genes by extension with a polymerase, usually a thermostable polymerase such as Taq, in a process that amounts to a slight variation of the polymerase chain reaction (PCR). The requirement for PCR-like conditions, however, imposes limits on the length of the genes that can be shuffled and can also be mutagenic.

To fragment genes, DNA shuffling requires stochastic digestion of DNA molecules with DNAseI. It is also possible to use restriction enzymes, however, such enzymes often produce fragments with cohesive ends that ligate to each other randomly rather than in the order in which they were initially connected. The restriction fragments alternatively could be assembled by PCR with the limitations discussed above. An additional difficulty with restriction enzymes is that the location of their restriction sites is random and would generally require the use and/or evaluation of many enzymes for useful fragmentation. Laborious optimization of restriction enzyme mixtures would be required for each new gene to be shuffled.

Another shortcoming of the aforementioned shuffling methods is that they are not amenable to single-stranded RNA systems. However, in certain cases it can be advantageous to work directly with RNA molecules. For example, many viral genomes consist of single strands of RNA, including flaviviruses such as Dengue, Japanese Encephalitis and West Nile, retroviruses such as HIV, and other animal and plant pathogens, including viroids (Fields et al., (1996) Fundamental Virology, 3^{rd} edition, Lippincott-Raven). By constructing recombinant viral genomes, valuable vaccines can be developed (see, for instance, Guirakhoo, et al., Virology, 1999, 257, 363-72 and Monath, et al., Vaccine, 1999, 17, 1869-82), and the availability of methods to do so more rapidly can accelerate this type of research. Assembly of full-length cDNA of a group I coronavirus using a series of smaller subclones has been reported in Yount, et al., J. of Virology, 2000, 10600.

An alternative DNA shuffling method is described in Coco, et al., Nature Biotechnology, 2001, 19, 354. The method involves the isolation of single-stranded forms of the genes to be shuffled as well as a complementary single-stranded template sequence. Providing such single-stranded species can be time-consuming and labor-intensive. The single-stranded DNA molecules are fragmented and assembled back into recombined sequences by hybridization to the complementary template. The various fragments aligned on any given template molecule are then fused into a single recombinant molecule by the action of a polymerase and a ligase. In order to improve the efficiency of this method, the single-stranded template must be degraded in the final step. This requires an additional step so that the single-stranded template is differentiated from the fragment molecules. This step involves replacing thymine bases with uracil so that the enzyme uracil N-glycosilase can destroy the template strand specifically.

For simplicity and ease, DNA shuffling methods currently rely on random DNA cleaving methods to prepare DNA fragments. However, techniques for site-directed cleavage of DNA are known, including techniques that do not require an artificially introduced restriction site. For instance, a method has been reported involving the cleaving of single-strands of DNA whereby an oligonucleotide adapter hybridizes to the polynucleotide strand and directs cleavage by a class IIS restriction enzyme between any two desired nucleotides (see, e.g., Kim, et al., Science, 1998, 240, 504-506, Podhajska, et al., Methods in Enzymology, 1992, 216, 303, Podhajska and Szybalski, Gene, 1985, 40, 175-82, Szybalski, Gene, 1985, 40, 169-73, and U.S. Pat. No. 4,935,357). Although, this “universal” restriction endonuclease has found great use in DNA sequencing and genomic mapping applications (see, for example, U.S. Pat. Nos. 5,710,000 and 6,027,894), indications that this technique might be used to productively generate recombined sequences are unknown. Class IIS restriction enzymes are also reported in “end selection” techniques related to the directed evolution methods of U.S. Pat. No. 6,238,884.

Current shuffling techniques generally recombine DNA via PCR-based methods. However, a non-shuffling method, involving the simultaneous mutation of multiple sites in a sequence, assembles mutant PCR fragments on a single-stranded DNA template and ligates the fragments by a ligase chain reaction (Weisberg, et al., Biotechniques, 1993, 15, 68-75). Fragmentation of mutant genes is carried out using the time-consuming process of PCR and agarose gel-purification. Additionally, there is no mention of how such fragmentation can be combined with the ligase chain reaction to achieve useful recombination of mutations. Moreover, the ligation efficiency of the method is low, due to the presence of large concentrations of complementary sequences that lead to the formation of blunt ends rather than the formation of ligatable nicks (see p. 74 in Weisberg et al., supra).

Current methods of in vitro recombination of DNA molecules are limited to polynucleotides of significant homology (>70% or >90%) and provide limited means of controlling recombination events. Also, using current methods of in vitro recombination, RNA molecules cannot be recombined directly. Moreover, these methods generally require the use of a polymerase to assemble fragments into recombined genes, thereby limiting the size of the DNA molecules that can easily be shuffled and increasing the mutagenicity of the process. For at least the above reasons, there exists a need for an alternative method of shuffling genes that allows less random recombination, avoids the use of a polymerase or PCR for assembly of shuffled genes, and can be applied readily to RNA molecules. The methods of the present invention, described herein, are directed toward this end.

### SUMMARY OF THE INVENTION

The present invention provides methods of preparing a library of polynucleotides. The methods comprise contacting a parent set of polynucleotides with at least one class IIS restriction enzyme to form a plurality of polynucleotide fragments. Members of the set of polynucleotides comprise at least one common class IIS restriction site capable of being cleaved by the at least one class IIS restriction enzyme. The method further comprises inactivating the at least one class IIS restriction enzyme, or separating the at least one class IIS restriction enzyme from said fragments. Additionally, the method comprises the step of ligating the fragments to yield full-length polynucleotides while allowing for the interchange of analogous fragments, thereby forming the library of polynucleotides.

The present invention includes a method of preparing a library of polynucleotides comprising: contacting a parent set of polynucleotides with a cleaving enzyme and at least one oligonucleotide adapter, wherein the oligonucleotide adapter directs cleavage of at least two polynucleotides within the set at homologous sites to form a plurality of polynucleotide fragments; ordering the fragments by hybridization with at least one template, allowing for the interchange of analogous fragments, wherein fragment ends resulting from cleavage using a common oligonucleotide adapter are adjacently positioned by the at least one template; and coupling the hybridized fragments to form the library of polynucleotides.

Further contemplated by the present invention is a method of preparing a library of polynucleotides comprising: contacting a parent set of polynucleotides with a restriction enzyme and at least one oligonucleotide adapter, wherein the adapter comprises a first region capable of hybridizing to at least one region of sequence homologous among the polynucleotide members and a second region comprising a recognition site for the restriction enzyme, wherein cleavage of the polynucleotides at homologous sites among the polynucleotides forms a plurality of polynucleotide fragments; ordering the fragments by hybridization with at least one template, allowing for the interchange of analogous fragments, wherein fragment ends resulting from cleavage using a common oligonucleotide adapter are adjacently positioned by the at least one template; and coupling the hybridized fragments to form the library of polynucleotides.

The present invention further embodies a method of preparing a library of polynucleotides comprising: contacting a parent set of RNA polynucleotides with a ribonuclease and at least one DNA oligonucleotide adapter to allow cleavage of the RNA polynucleotides at homologous sites, forming a plurality of RNA polynucleotide fragments; ordering the fragments by hybridization with at least one template, allowing for the interchange of analogous fragments, wherein fragment ends resulting from cleavage using a common oligonucleotide adapter are adjacently positioned by the at least one template; and coupling the hybridized fragments to form the library of polynucleotides.

Also provided by the present invention are libraries of polynucleotides prepared by any of the methods described above.

Other embodiments of the present invention include a method of preparing a polynucleotide with a predetermined property, comprising generating a library of polynucleotides according to any of the methods described above, and identifying at least one polynucleotide within the library having the predetermined property.

The present invention also includes methods of preparing a polynucleotide with a predetermined property, comprising generating a library of polynucleotides according to any of the methods described above; identifying at least one polynucleotide within the library having the predetermined property; and repeating the generating and identifying steps wherein at least one fragment of the identified polynucleotides is preferentially incorporated into the library.

### BRIEF DESCRIPTION OF THE DRAWINGS

FIG. 1 illustrates an embodiment of the present invention

### DETAILED DESCRIPTION OF PREFERRED EMBODIMENTS

In general, the present methods can be described as the recombination of polynucleotides by fragmentation of polynucleotide strands, interchange of analogous strand fragments, and ligation of interchanged strand fragments.

As used herein, the term “polynucleotide” means a polymer of nucleotides including ribonucleotides and deoxyribonucleotides, and modifications thereof, and combinations thereof. Preferred nucleotides include, but are not limited to, those comprising adenine (A), guanine (G), cytosine (C), thymine (T), and uracil (U). Modified nucleotides include, but are not limited to, those comprising 4-acetylcytidine, 5-(carboxyhydroxylmethyl)uridine, 2-O-methylcytidine, 5-carboxymethylaminomethyl-2-thiouridine, 5-carboxymethylamino-methyluridine, dihydrouridine, 2-O-methylpseudouridine, 2-O-methylguanosine, inosine, N6-isopentyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, 5-methoxyuridine, 5-methoxycarbonylmethyl-2-thiouridine, 5-methoxycarbonylmethyluridine, 2-methylthio-N6-isopentyladenosine, uridine-5-oxyacetic acid-methylester, uridine-5-oxyacetic acid, wybutoxosine, wybutosine, pseudouridine, queuosine, 2-thiocytidine, 5-methyl-2-thiouridine, 2-thiouridine, 4-thiouridine, 5-methyluridine, 2-O-methyl-5-methyluridine, 2-O-methyluridine, and the like. The polynucleotides of the invention can be single-stranded or double-stranded, and can also comprise both ribonucleotides and deoxyribonucleotides in the same polynucleotide. Polynucleotides can have phosphodiester backbones or modified backbones such as, for example, phosphorothioate. Polynucleotides can also comprise genes, gene fragments, and the like, and can be of any length. Polynucleotide length can range from about 200 to about 20,000 nucleotides, or more. According to some embodiments, polynucleotide length ranges from about 200 to about 10,000, about 200 to about 8000, about 200 to about 5000, about 200 to about 3000, or about 200 to about 1000 nucleotides. In other embodiments, polynucleotide length can range from about 200 to about 2000, about 2000 to about 5000, about 5000 to about 10,000, about 10,000 to about 20,000, or greater than 20,000 nucleotides.

As used herein, the term “oligonucleotide” means a polymer of nucleotides, including ribonucleotides and deoxyribonucleotides, and modifications thereof, and combinations thereof, as described above. Oligonucleotides can range from about 2 nucleotides to about 200 nucleotides, from about 20 nucleotides to about 100 nucleotides, or about 40 to about 60 nucleotides. Oligonucleotides of any predetermined sequence comprising DNA and/or RNA are readily accessible, such as by synthesis on a nucleic acid synthesizer. Other methods for their syntheses and handling are well known to those skilled in the art.

The term “library,” as used herein, refers to a plurality of polynucleotides or polypeptides in which the members have different sequences. “Combinatorial library” indicates a library prepared by combinatorial methods. In general, libraries of polynucleotides comprise a plurality of different polynucleotides, typically generated by randomization or combinatorial methods that can be screened for members having desirable properties. Libraries can comprise a minimum of two unique members but typically, and desirably, contain a much larger number. Larger libraries are more likely to have members with desirable properties, however, current screening methods have difficulty handling very large libraries (i.e., of more than a few thousand unique members). Thus, libraries can comprise from about 10¹ to about 10¹⁰, or from about 10² to about 10⁵, or from about 10³ to about 10⁴ unique polynucleotide members.

The phrase “parent set of polynucleotides” means a set of at least two different polynucleotide members. Polynucleotide members of the parent set need not be related by homology or any other criterion. In some embodiments, however, polynucleotide members of the parent set are related by homology at the nucleotide and/or amino acid level. Any level of homology is suitable, however, homologies include at least about 65%, at least about 70%, at least about 75%, at least about 80%, at least about 85%, at least about 90%, at least about 95%, and at least about 99% percent identity at either the nucleotide or amino acid level. Homology can be determined using the computer program BLAST with default parameters, publically available on-line at www.ncbi.nlm.nih.gov/BLAST/. Polynucleotide members can be single-stranded or double-stranded.

Further, in some embodiments, the parent set of polynucleotides can be selected according to their function. As a non-limiting example, one or more polynucleotide sequences can be identified from public sources, such as literature databases (e.g., PubMed), sequence databases like (e.g., GenBank), or enzyme databases available on-line from ExPASy of the Swiss Institute of Bioinformatics, based on their ability to code for proteins capable of catalyzing a certain chemical reaction. Upon identification of a polynucleotide, others sharing homology at the nucleotide or amino acid level can be further identified using homology searching tools, such as BLAST.

The basis for selection of a set of parent polynucleotides can be a specific property, function, or physical characteristic that is desirable in the recombined sequences of the library. For instance, if a recombined polynucleotide sequence capable of coding for an enzyme that catalyzes a reaction at high pH is desired, then of the possible polynucleotide sequences that catalyze the reaction, only the ones that perform at high pH are selected to comprise the parent set of polynucleotides. In another approach to making sets of polynucleotides that makes fewer assumptions about the contribution of sequence to phenotype and allows for greater diversity, members of the set can be chosen according to phylogenies. For example, a set of polynucleotides sharing a predetermined minimal sequence homology can be organized into a phylogenetic tree. Algorithms allowing the assembly of homologous sequences into phylogenetic trees are well known to those skilled in the art. For instance, the phylogenetic tree building program package Phylip is readily available to the public on-line at evolution.genetics.washington.edu/phylip.html maintained by the University of Washington. Sequences representing different branches of the calculated phylogenetic tree can then be selected to comprise a set of polynucleotides.

As used herein, the term “cleaving enzyme” is meant to refer to an enzyme that is capable of cleaving polynucleotides. Cleaving enzymes include, but are not limited to, restriction enzymes and nucleases. Restriction enzymes include class IIS restriction enzymes. This class of enzymes differs from other restriction enzymes in that the recognition sequence is separate from the site of cleavage. In this respect, the resulting cohesive ends are less likely to be palindromic, a condition that would lead to undesirable scrambling of fragments during reassembly. Some examples of class IIS resctriction enzymes include AlwI, BsaI, BbsI, BbuI, BsmAI, BsrI, BsmI, BspMI, Earl, Esp3I, FokI, HgaI, HphI, MboII, PleI, SfaNi, MnlI, and the like. Many of these restriction enzymes, such as FokI, are available commercially and are well known to those skilled in the art. Nucleases suitable for the present invention are capable of cleaving polynucleotides at DNA/RNA heteroduplex regions. Nucleases include ribonucleases such as, but not limited to, RNase H and the like.

As used herein, the term “contacting” means the bringing together of compounds to within distances that allow for intermolecular interactions and/or transformations. “Contacting” can occur in the solution phase.

The term “coupling,” as used herein, means the covalent linking of molecules. Coupling of polynucleotides, oligonucleotides, and/or fragments thereof can be carried out using a ligase, such as, for example, a DNA ligase or an RNA ligase. “Ligating” refers to the coupling of polynucleotides, oligonucleotides, and/or fragments using a ligase.

As used herein, the phrase “oligonucleotide adapter” is meant to refer to a single-stranded oligonucleotide capable of hybridizing to a polynucleotide and directing enzymatic cleavage of the polynucleotide. An oligonucleotide adapter directs enzymatic cleavage by creating a cleavage site recognizable by a cleaving enzyme upon hybridization of the adapter to a polynucleotide. When the nucleotide sequence of an adapter is designed to be complementary to a portion of target polynucleotide (i.e., the polynucleotide undergoing cleavage) in such a way that it directs enzymatic cleavage between two desired nucleotides in the target polynucleotide, the oligonucleotide adapter is referred to as “defined.” Alternatively, when the adapter comprises a random sequence to facilitate cleavage at random sites in a target polynucleotide, the oligonucleotide adapter is referred to as “random.” In some embodiments, the oligonucleotide adapter comprises a first region and a second region. The first region is preferably capable of hybridizing to a target polynucleotide and the second region comprises a recognition site for a restriction enzyme. Design and synthesis of oligonucleotide adapters comprising restriction enyzme recognition sites and their use in directed cleavage of DNA is reported in Kim, et al., Science, 1998, 240, 504-506, Podhajska, et al., Methods in Enzymology, 1992, 216, 303, Podhajska and Szybalski, Gene, 1985, 40, 175-82, Szybalski, Gene, 1985, 40, 169-73, and U.S. Pat. No. 4,935,357, each of which is incorporated herein by reference in its entirety.

As used herein, the term “common” means similar or the same. Thus, a “common” oligonucleotide adapter facilitates polynucleotide cleavage at homologous sites among a set of different polynucleotides. Accordingly, a restriction site is “common” among members of a plurality of polynucleotides when it is cleavable by the same restriction enzyme and located substantially in the same region of sequence in each member.

The term “homologous,” as used herein, means similar or having a degree of homology. In particular, polynucleotide regions or sites that are “homologous” correspond to regions of sequence that have relatively high sequence identity. Percent identities for homologous regions of sequence can include at least about 65%, at least about 70%, at least about 75%, at least about 80%, at least about 85%, at least about 90%, and at least about 95% percent identity.

As used herein, the term “fragment” is meant to refer to a segment of single-stranded or double-stranded polynucleotide generated by cleaving a polynucleotides with a cleaving enzyme. “Analogous fragments” are fragments from different polynucleotides that are the result of cleavage at a site common to the different polynucleotides. To illustrate, different polynucleotides, each having a common restriction site along the sequence at a certain position x from the 5′ end, are cleaved. Fragments from the different polynucleotides containing the 5′ end and an end resulting from cleavage at x are analogous. Similarly, fragments from different polynucleotides containing the 3′ end and an end resulting from cleavage at x are also analogous. Analogous fragments can be, but are not necessarily, homologous.

As used herein, the term “template” refers to a single-stranded polynucleotide or oligonucleotide having a predetermined sequence that comprises regions of complementarity with at least two polynucleotide fragments. Templates facilitate the ordering and coupling of fragments by hybridization. One or more templates can be used to assemble a full-length polynucleotide. Templates designed to facilitate the ordering and coupling of polynucleotide fragments in systems of relatively low homology, such as, for example, less than about 70% percent identity, are referred to as “bridging oligonucleotides.” Templates can also be intrinsic to the fragmented polynucleotide system, whereby fragments themselves can serve as templates. As such, fragments that also serve as templates share regions of complementarity with other fragments, and can be generated, for example, by fragmenting double stranded DNA in such a way that enzymatic cleavage results in nicks in one or both strands, each nick occurring at a unique site in the sequence.

As used herein, the term “screening” or “screen” refers to processes for assaying large numbers of library members for a predetermined or desired characteristic. Characteristics include any distinguishing property of a polynucleotide or polypeptide including, but not limited to, structural characteristic, enzymatic activity, or ligand binding affinity.

As used herein, the phrase “predetermined property” refers to a polynucleotide or polypeptide characteristic that is assayed or tested. “Predetermined properties” include any distinguishing characteristic, such as structural or functional characteristics, of a polynucleotide or polypeptide including, but not limited to, primary structure, secondary structure, tertiary structure, encoded enzymatic activity, catalytic activity, stability, or ligand binding affinity. Some predetermined properties pertaining to enzyme and catalytic activity include higher or lower activities, broader or more specific activities, and activity with previously unknown or different substrates relative to wild type. Some predetermined properties related to ligand binding include, but are not limited to, weaker or stronger binding affinities, increased or decreased enantioselectivities, and higher or lower binding specificities relative to wild type. Other predetermined properties can be related to the stability of proteins, including enzymes, with respect to organic solvent systems, cofactors, temperature, and sheer forces (i.e., stirring and ultrafiltration). Further, predetermined properties can be related to the ability of a protein to function under certain conditions related to temperature, pH, salinity, and the like. Predetermined properties are often the goal of directed evolution efforts in which a protein or nucleic acid is artificially evolved to exhibit new and/or improved properties relative to wild type.

Certain embodiments of the present invention include methods for the preparation of libraries of polynucleotides involving the shuffling of a parent set of polynucleotide molecules having either or both native and engineered class IIS restriction sites. For example, the polynucleotide members of the parent set, that share at least one class IIS restriction site can be contacted with one or more corresponding class IIS restriction enzymes for a time and under conditions sufficient to cleave the parent polynucleotides to yield fragments. Because of the nature of the fragment ends (non-palindromic) generated by cleavage with a class IIS restriction enzyme, the fragments can be ligated back together in the correct order, while allowing for fragment interchange or “shuffling.” In this way, a library of polynucleotides can be produced having greater diversity than the parent set.

Any polynucleotide is suitable for the above method, including those without native class IIS restriction sites. For polynucleotides in which there are no native class IIS restriction sites, a modified version of the gene can be designed to include the desired restriction sites without altering the encoded amino acid sequence. Likewise, genes containing more than the desired number of class IIS restriction sites, or unwanted restriction sites that would result in undesirable (e.g., palindromic) cohesive ends, can be modified to contain fewer such sites. Methods for modification of polynucleotides are well known to the skilled artisan and can include site-directed mutagenesis or other techniques.

According to some embodiments, such as in cases where the parent polynucleotides contain restriction sites corresponding to different class IIS restriction enzymes, more than one class IIS restriction enzyme can be used. Enzymes that produce cohesive ends having overhangs are particularly suitable. Overhangs of at least 2, 3, 4 or more nucleotides, can be appropriate for carrying out the above methods. Longer overhangs facilitate correct ordering of the fragments upon ligation.

Standard non-class IIS restriction enzymes such as EcoRI or BamHI would not be appropriate for carrying out the above procedures because their palindromic cohesive ends would not facilitate assembly of the fragments in their original order. Moreover, the present method is advantageous over previous methods because the achieved fixed cross-over recombination frequency can be as high as theoretically possible, in contrast with other methods which suffer from lower frequencies (see, e.g., Pelletier, Nature Biotechnology, 2001, 19, 314).

The above method can also include the use of oligonucleotide adapters to direct cleavage of the parent set of polynucleotides. The adapters can be designed to hybridize to specific regions common among the parent set to allow cleavage by class IIS restriction enzymes. Using the adapter, cleavage can be directed to occur between specific pairs of bases in the parent polynucleotides. In this way, the need to modify the parent polynucleotides to include restriction sites, additional to any native restriction sites, is reduced.

An example of a method according to the present invention using oligonucleotide adapters includes the step of contacting a parent set of polynucleotides, such as, for example, at least two different double-stranded DNA molecules, and at least one oligonucleotide adapter capable of directing cleavage of the DNA molecules with a class IIS restriction enzyme, such as FokI, for example. While not wishing to be bound by theory, it is believed that when the parent polynucleotides are hybridized with the adapter, the class IIS restriction enzymes cleave the parent polynucleotides, thereby creating nick sites. According to some embodiments, a plurality of different oligonucleotide adapters can be contacted with the parent set of polynucleotides, each adapter being specifically designed to target different regions of sequence, including both sense and anti-sense strands. Adapters can be designed to place nick sites along the length of double-stranded parent polynucleotides in an alternating fashion, alternating between sense and anti-sense strands.

The method further includes the step of separating the nicked DNA (or fragmented strands) from the oligonucleotide adapters and from the restriction enzyme. Any technique known in the art can be used to effect the separation. For example, the fragmented DNA can be purified (e.g., by purification with a Qiaquick PCR purification kit (Qiagen, Inc.)). It can also be sufficient to inactivate the enzyme by heat treatment, such as, for example, the same heat treatment used to melt the fragmented strands in a subsequent step.

The method also includes melting and reannealing of the fragmented strands to allow interchange (or reassortment) of fragments. The melting and reannealing can be repeated any number of times until sufficient interchange is obtained. The method further includes the step of contacting the resulting reannealed duplexes (whether they be heteroduplexes or homoduplexes) with a ligase to repair the nicks, thereby generating the desired library of polynucleotides. Any suitable ligase, such as a DNA ligase, can be used. In further embodiments, the melting and reannealing steps, as well as the contacting with a ligase can be optionally repeated until full-length DNA is obtained and/or a useful amount of full-length DNA is available for further procedures such as, for example, amplification or molecular cloning. Gel electrophoresis or any other appropriate technique can be used to detect recombined full-length DNA.

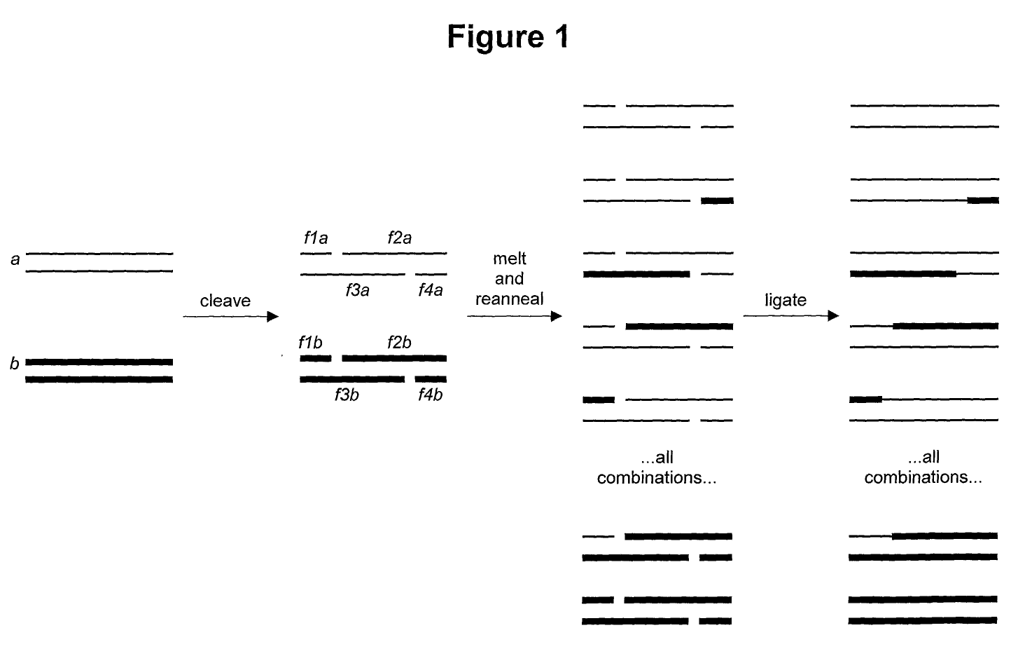

FIG. 1 depicts an embodiment of the present invention for illustrative purposes. A parent set of double-stranded DNA is represented by polynucleotides a and b having at least about 90% homology at the nucleotide level. Two different oligonucleotide adapters are introduced (not shown), each of which is designed to direct cleavage at different homologous sites, one in an upper strand and one in a lower strand. Upon cleavage with an appropriate enzyme, each double-stranded polynucleotide is broken into four fragments (i.e., f1a, f2a, f3a, and f4a). The analogous fragments (i.e., f1a and f1b) can then be interchanged (or reassorted) by melting and annealing (several cycles if necessary). Since each of the fragments share complementarity with at least one other fragment, the fragments serve as templates during annealing so that they are reassembled in the correct order. The reassorted and annealed fragments are then ligated using a DNA ligase. Of the possible number of double-stranded results, a total of four new chimeric polynucleotides are prepared (not including their complements), represented as f1a+f2b, f1b+f2a, f3a +f4b, and f3b+f4a.

Although homology of at least about 70%, 75%, 80%, 85% or least about 90% is particularly suitable, the present invention includes methods for the preparation of libraries from a parent set of non-homologous polynucleotides, or polynucleotides related by low homology, such as, for example, less than about 70% homology. For example, two sequences (referred to as c and d) of low homology can be recombined by using oligonucleotide adapters that recognize sequence regions between which a recombination event is to occur, as selected by the person carrying out the procedure. In the melting and reannealing step, as discussed above, bridging oligonucleotides can be introduced which align and permit ligation of a fragment of sequence c with another fragment of sequence d to yield a chimeric molecule comprising a section of sequence c followed by a section of sequence d, or vice versa. Additional sequences can be recombined in this manner by adding oligonucleotide adapters and bridging oligonucleotides as necessary.

In other embodiments, the present invention includes methods of shuffling RNA molecules. The methods generally include the cleavage of a parent set of RNA molecules at heteroduplex regions formed by hybridization of DNA oligonucleotides to the RNA. Cleavage is effected by treatment with an RNAse H enzyme that targets the heteroduplex regions and cuts the RNA to form RNA fragments. The resulting hybridized fragments can be melted and reannealed to effect swapping of RNA fragments while retaining sequence order. Ligation of the fragmented RNA results in a library of RNA molecules having greater diversity than the parent set.

To illustrate, the methods comprise the step of contacting at least one complementary DNA oligonucleotide to members of a parent set of RNA molecules, forming at least one homologous heteroduplex region common among members of the parent set. Any number of different DNA oligonucleotides can be used and designed to target any desired region of RNA for cleavage. In some embodiments, 1, 2, 3, 4, 5, 6, 7, 8, 9, 10 or more different DNA oligonucleotides can be used, for example. Complementary DNA oligonucleotides of at least 4, 5, 6, 7, 8, 9, 10 or more nucleotides are appropriate for the present methods.

The methods further comprise the step of contacting the resulting RNA:DNA heteroduplexes with RNAse H to effect cleavage of the RNA and generate RNA fragments. Cleavage of the RNA molecules can be directed to one phosphodiester bond in the region of, or immediately adjacent to, the heteroduplex region. According to some embodiments, cleavage of RNA by RNAse H can be directed to a site at the 5′ end of the hybridized DNA oligonucleotide as described in Donis-Keller, Nucleic Acids Research, 1979, 7, 179, which is incorporated herein by reference in its entirety. Accordingly, DNA oligonucleotides can be designed to effect cleavage of the RNA molecules at specific sites.

The methods further comprise the step of removing or inactivating RNAse H after cleavage of the RNA molecules. Any method of inactivation or removal is suitable. For example, RNAse H can be inactivated by heat treatment. In a subsequent step, the methods involve the melting and reannealing of the generated RNA fragments with bridging oligonucleotides so as to maintain fragment order. The DNA oligonucleotides used during cleavage can also serve as the bridging oligonucleotides. In other embodiments, bridging oligonucleotides can be different from those used to direct cleavage. The melting and reannealing can be repeated any number of times until sufficient fragment mixing is obtained.

In a further step, the reannealed RNA fragments can be ligated to form a library of RNA molecules having greater diversity than the parent set. Ligation can be carried out using a ligase according to known methods. DNA ligases, such as T4 DNA ligase, are suitable. Any remaining hybridized DNA oligonucleotides and/or bridging oligonucleotides can be removed by typical nucleic acid purification techniques. The resulting recombinant RNA molecules can be assayed directly or reverse-transcribed by RT-PCR to generate recombined DNA molecules.

Once generated, libraries of polynucleotides can be manipulated directly, or can be inserted into appropriate cloning vectors and expressed. Methods for cloning and expression of polynucleotides, as well as libraries of polynucleotides, are well known to those skilled in the art.

Libraries of polynucleotides, or the expression products thereof, can be screened for members having desirable new and/or improved properties. Any screening method that can result in the identification or selection of one or more library members having a predetermined property or desirable characteristic is suitable for the present invention. Methods of screening are well known to those skilled in the art and include, for example, enzyme activity assays, biological assays, or binding assays. Screening methods include, but are not limited to, phage display and other methods of affinity selection, including those applied directly to polynucleotides. Other preferred methods of screening involve, for example, imaging technology and colorimetric assays. Suitable screening methods are further described in Marrs, et al., Curr. Opin. Microbiol., 1999, 2, 241; Bylina, et al., ASM News, 2000, 66, 211; Joyce, G. F., Gene, 1989, 82, 83; Robertson, et al., Nature, 1990, 344, 467; Chen, et al., Proc. Natl. Acad. Sci. USA, 1993, 90, 5618; Chen, et al., Biotechnology, 1991, 9, 1073; Joo, et al., Chem. Biol., 1999, 6, 699; Joo, et al., Nature, 1999, 399, 670; Miyazaki, et al., J. Mol. Evol., 1999, 49, 716; You, et al., Prot. Eng., 1996, 9, 77; and U.S. Pat. Nos. 5,914,245 and 6,117,679, each of which is incorporated herein by reference in its entirety.

Polynucleotides identified by screening of a library can be readily isolated and characterized. Characterization includes sequencing of the identified polynucleotides using standard methods known to those skilled in the art.

In some embodiments of the present invention, a recursive screening method can be employed for preparing or identifying a polynucleotide with a predetermined property from a library. An example of a recursive screening method is recursive ensemble mutagenesis described in Arkin, et al., Proc. Natl. Acad. Sci. USA, 1992, 89, 7811; Delagrave, et al., Protein Eng., 1993, 6, 327; and Delagrave, et al., Biotechnology, 1993, 11, 1548, each of which is herein incorporated by reference in its entirety. According to this method, one or more polynucleotides, having a predetermined property, are identified from a first library by a suitable screening method. The identified polynucleotides are characterized and the resulting information used to assemble a further library. For instance, one or more fragments of the identified polynucleotides can be preferentially incorporated into a further library which can also be screened for polynucleotides with a desirable property. Methods for the isolation of fragments for incorporation into further libraries is well known to those skilled in the art. In some embodiments, all fragments of an identified polynucleotide can be incorporated into the further library by including the identified polynucleotide itself into the parent set. Generating a library by incorporating the fragments identified from a previous cycle can be repeated as many times as desired. The recursion can be terminated upon identification of one or more library members having a predetermined or desirable property that is superior to the desirable property of the identified polynucleotides of previous cycles or that meets a certain threshold or criterion. According to this method, fragments that do not lead to functional sequences are eliminated from the pool of oligonucleotides used to generate the next library generation. Furthermore, amounts of fragments used in the preparation of a further library can be weighted according to their frequency of occurrence in the identified polynucleotides. Alternatively, if the identified polynucleotides are too small in number to accurately represent the true frequency of occurrence in a population of desirable polynucleotides, their amounts can be equally weighted. As an example, if the initial set of polynucleotides was chosen based on equal representation of branches of a phylogenetic tree, it is possible that certain families would be represented more frequently than others in the polynucleotides identified with a screen. Thus, polynucleotides belonging to these families but not used in the initial generation of a library can be used to prepare a further library generation, thus expanding diversity while preserving a bias towards desirable sequences.

Collectively, the methods of the present invention allow for rapid and controlled “directed evolution” of genes and proteins. The present methods facilitate the preparation of biomolecules having desirable properties that are not naturally known or available. Uses for these improved biomolecules are widespread, promising contributions to the areas of chemistry, biotechnology, and medicine. The methods of the present invention can be used, for example, to prepare enzymes having improved catalytic activities and receptors having modified ligand binding affinities, to name a few, are just some of the possible achievements of the present invention.

Those skilled in the art will appreciate that numerous changes and modifications can be made to the embodiments of the invention described herein and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated by reference in their entireties.

As illustrated in Examples 1 and 2, by varying the sequence of oligonucleotide adapters, cleavage can be made random or directed to specific sites along the sequences of the genes to be shuffled. Thus greater control of recombination sites and frequency is afforded by the present method. Example 3 illustrates RNA shuffling. Examples 4-6 provide experimental details and results for shuffling of galactose oxidase mutants. Examples 1-3 are prophetic and Examples 4-6 are actual.

### EXAMPLES

### Example 1

### Shuffling of Galactose Oxidase (GO) Mutants Using Defined Oligonucleotide Adapters

Mutants of the enzyme galactose oxidase (GO), generated as described in Delagrave, et al., Protein Engineering, 2001, 14, 261 and U.S. Pre-grant Publication No. 20010051369, each of which is incorporated herein by reference in its entirety, are chosen to be shuffled in order to create new mutants carrying new combinations of mutations: the wildtype GO clone (GOK3) as well as clones GO8-1H3A and 7.3.2.

Oligonucleotides comprising a hairpin loop containing a FokI recognition sequence and a region of complementarity to 14 nt regions of the GO gene are prepared. The oligonucleotides are complementary to either the top or bottom strand of the GO open reading frame (ORF) in an alternating sequence along the length of the ORF. The oligonucleotide binding sites are chosen to be spaced roughly 120 bp from each other. Oligonucleotides having binding sites that would fall within about 50 bp of a native FokI site in the gene are omitted.

List of oligonucleotides: (FokI-binding site hairpin loop is upper case, sequence complementary to GO is lower case.)

| | |
|---|---|
| GOFok1 | |
| 5′-CACATCCGTGCACGGATGTGtcctgagccttcg | (SEQ ID NO:1) |
| agcct-3′ | |
| GOFok2 | |
| 5′-gtgtgccaaaaggtatCACATCCGTAGGATGTG | (SEQ ID NO:2) |
| -3′ | |
| GOFok3 | |
| Near native FokI site, no oligo- | |
| nucleotide necessary. | |
| GOFok4 | |
| 5′-gcagggcgagtttcaaCACATCCGTAGGATGTG | (SEQ ID NO:3) |
| -3′ | |
| GOFok5 | |
| 5′-CACATCCGTGCACGGATGTGctggtcttggacg | (SEQ ID NO:4) |
| ctgg-3′ | |
| GOFok6 | |
| 5′-gatcccctggtggtatcCACATCCGTAGGATGT | (SEQ ID NO:5) |
| G-3′ | |
| GOFok7 | |
| Near native FokI site, no oligo- | |
| nucleotide necessary. | |
| GOFok8 | |
| 5′-ccgtctgacatggtagCACATCCGTAGGATGTG | (SEQ ID NO:6) |
| -3′ | |
| GOFok9 | |
| 5′-CACATCCGTGCACGGATGTGaggtcaacccaat | (SEQ ID NO:7) |
| gttg-3′ | |
| GOFok10 | |
| 5′-ccactggtatagtaccCACATCCGTAGGATGTG | (SEQ ID NO:8) |
| -3′ | |
| GOFok11 | |
| 5′-CACATCCGTGCACGGATGTGtcctgacctttgg | (SEQ ID NO:9) |
| cgg-3′ | |
| GOFok12 | |
| 5′-gaaacgtcgggcaaagtCACATCCGTAGGATGT | (SEQ ID NO:10) |
| G-3′ | |
| GOFok13 | |
| 5′-CACATCCGTGCACGGATGTGacgtccctgaaca | (SEQ ID NO:11) |
| agac-3′ | |
| GOFok14 | |
| 5′-gattcgtggtacaatcgcCACATCCGTAGGATG | (SEQ ID NO:12) |
| TG-3′ | |
| GOFok15 | |
| 5′-CACATCCGTGCACGGATGTGtcggcggccgcat | (SEQ ID NO:13) |
| tac-3′ | |
| GOFok16 | |
| 5′-gctatttcctccattgtCACATCCGTAGGATGT | (SEQ ID NO:14) |
| G-3′ | |

PCR products from each clone are generated according to standard methods using primers badmcssns (5′-CTACTGTTTCTCCATACCCG-3′; SEQ ID NO: 15) and badmcsant (5′-AAACAGCCAAGCTGGAGACC-3′; SEQ ID NO: 16).

The 3 PCR products are gel-purified using a Qiagen gel extraction kit and mixed in equal molar amounts such that the final concentration of DNA, in a final volume of 20 to 30 μL, is ˜30 ng/μL in a buffer containing 20 mM KCl, 10 mM Tris-HCl, pH 7.5, 10 mM MgCl₂, 0.5 mM DTT and 12 pmol of each of the 15 GOFok oligonucleotides listed above (a 15-fold molar excess of each primer compared to the PCR DNA). This mixture is heated to 95° C. for 1.5 minutes and rapidly cooled to 37° C.

At least 12 units of the enzyme FokI (New England Biolabs) are added to the cooled mixture. The resulting solution is allowed to incubate 5 min to 3 hours at 37° C. Aliquots of the reaction can be analyzed by agarose gel electrophoresis or denaturing polyacrylamide gel electrophoresis to determine the extent of FokI digestion.

Following the incubation, the digested fragments are separated from the enzyme and oligonucleotides by the use of a Qiagen PCR purification kit. The purified gene fragments are eluted from the Qiagen purification column using H₂O or dilute TE buffer in a volume of 40 μL, as prescribed by the kit protocol.

A 4.4 μL aliquot of 10× ligation buffer (Roche) is added to the gene fragment solution and the resulting solution is heated to 95° C. for 1.5 minutes and cooled slowly (e.g., over 1 hour) to 25° C. At least ten units of T4 DNA ligase (Roche) are added and the solution is allowed to incubate for at least 1 hour at 25° C. Progress of the ligation can be monitored using an agarose gel.

When the desired ˜2 kb gene product is observed, it is cloned into the original expression vector, according to standard methods and as described in Delagrave, et al., Protein Engineering, 2001, 14, 261 or U.S. Pre-grant Publication No. 20010051369, each of which is incorporated herein by reference in its entirety. If the amount of gene product is too small to clone conveniently, it can be amplified by PCR according to standard methods prior to attempting cloning.

The resulting library of shuffled GO mutants is screened, also according to Delagrave, et al., Protein Engineering, 2001, 14, 261 and U.S. Pre-grant Publication No. 20010051369, each of which is incorporated herein by reference in its entirety, for a property of interest such as the ability to oxidize guar at elevated temperatures. Mutants showing improved properties are isolated and further characterized.

### Example 2

### Shuffling of Galactose Oxidase (GO) Mutants Using Random Oligonucleotide Adapters

Mutants of the enzyme galactose oxidase (GO), generated as described by Delagrave, et al., Protein Engineering, 2001, 14, 261 and U.S. Pre-grant Publication No. 20010051369, are chosen to be shuffled in order to create new mutants carrying new combinations of mutations: the wildtype GO clone (GOK3) as well as clones GO8-1H3A and 7.3.2.

A degenerate oligonucleotide comprising a hairpin loop containing the FokI recognition sequence and a region of random sequence is prepared according to standard methods, or ordered from a custom oligonucleotide supplier such as Operon Inc.

FokN6: 5′-CACATCCGTGCACGGATGTGNNNNNN-3′ (SEQ ID NO: 17)

A less complex oligonucleotide (e.g., FokN3: 5′-CACATCCGTGCACGGATGTGATGNNN-3′ (SEQ ID NO: 18) could also be used, resulting in a more restricted range of cleavage sites along the sequences, thereby facilitating the assembly step.

PCR products from each clone are generated according to standard methods using primers badmcssns and badmcsant (sequences provided in Example 1).

The 3 PCR products are gel-purified using a Qiagen gel extraction kit and mixed in equal molar amounts such that the final concentration of DNA, in a final volume of 20 to 30 μL, is ˜30 ng/μL in a buffer containing 20 mM KCl, 10 mM Tris-HCl, pH 7.5, 10 mM MgCl_{2, 0.5} mM DTT and at least 12 pmol of the FokN6 oligonucleotide listed above (a 15-fold molar excess of primer compared to the PCR DNA). This mixture is heated to 95° C. for 1.5 minutes and rapidly cooled to 37° C.

At least 12 units of the enzyme FokI (New England Biolabs), at least 1 unit of the Klenow fragment (3′→5′ exo-) (New England Biolabs) and dATP+dCTP+dGTP (to a final concentration of 33 μM each) are added to the cooled mixture. The resulting solution is allowed to incubate from 5 minutes to 3 hours at 37° C. Aliquots of the reaction can be analyzed by agarose gel electrophoresis or denaturing polyacrylamide gel electrophoresis to determine the extent of FokI digestion. Digestion should only proceed to an extent where most of the fragments are at least 200 to 300 nt in length.

Following the incubation, the digested fragments are separated from the enzymes and oligonucleotides by the use of a Qiagen PCR purification kit. The purified gene fragments are eluted from the Qiagen purification column using H₂O or dilute TE buffer in a volume of 40 μL, as prescribed by the kit protocol.

A 4.4 μL aliquot of 10× Ampligase buffer (Epicentre Technologies Inc.) and 10 to 50 units of Ampligase (Epicentre Technologies Inc.) are added added to the gene fragment solution. The resulting solution is heated to 95° C. for 1.5 minutes, cooled rapidly to 45° C. and allowed to incubate at that temperature for 4 minutes. This cycle of heating and cooling can be performed in a thermal cycler (e.g., 9700 from Applied Biosystems Inc.) and repeated numerous times (e.g., from 5 to 40 times) until the desired ligation product is observed. Progress of the ligation can be monitored using an agarose gel.

When the desired ˜2 kb gene product is observed, it is cloned into the original expression vector, according to standard methods and as described in Delagrave, et al., Protein Engineering, 2001, 14, 261 or U.S. Pre-grant Publication No. 20010051369. If the amount of gene product is too small to clone conveniently, it can be amplified by PCR according to standard methods prior to attempting cloning.

The resulting library of shuffled GO mutants is screened, also according to Delagrave, et al., Protein Engineering, 2001, 14, 261 and U.S. Pre-grant Publication No. 20010051369, for a property of interest such as the ability to oxidize guar at elevated temperatures. Mutants showing improved properties are isolated and further characterized.

### Example 3

### Shuffling of Flavivirus RNA Genomes

The genome of yellow fever 17D vaccine strain is isolated from the culture supernatant of infected cells containing high titers of virus according to standard techniques. The genome of Japanese encephalitis SA14-14-2 is similarly isolated.

In a 20 μL volume, equal amounts of each genome (˜100 ng) are mixed together and with a molar excess of cleavage oligonucleotides of defined sequence complementary to regions of the sequence where recombination is to occur. To the mixture is also added a 2.2 μL aliquot of 10× RNAse H buffer (Epicentre Technologies Inc.) and the resulting solution is heated to 60° C. for 3 minutes. RNAse H (Epicentre Technologies, 0.2 to 1.5 units) is added and the solution is brought to 37° C. for 30 minutes or for as long as is necessary to cleave the majority of RNA strands.

RNA is then extracted from the resulting solution, thereby separating it from enzyme, oligonucleotides and other buffer components, using an RNA purification kit as can be purchased from Qiagen Inc.

The resulting RNA solution (30 μL) is mixed with 3.3 μL of 10× T4 DNA ligase buffer and with a molar excess of oligonucleotides complementary to the desired chimeric sequence-junctions (bridging oligonucleotides). The mixture is heated to 60° C. for 3 minutes. T4 DNA ligase (Roche, 1 to 15 units) is added and the solution is brought to 37° C. for 30 minutes or for as long as is necessary to ligate the majority of RNA strands.

The recombined RNA molecules are then transfected into an appropriate cell line and viable recombinant viral genomes will be packaged by the cells into viral particles that are released into the growth medium. These recombinants can be plaque-purified according to standard methods and assayed for a desired property such as the ability to confer immunity to virulent strains of Japanese encephalitis.

### Example 4

### Shuffling of Galactose Oxidase (GO) Mutants Using Defined Sequence Adapter Oligonucleotides

Plasmids pBADGOK3 (K3) and pBADGO8-1 (8-1) were used as templates to amplify the 2 kb GO ORF by PCR according to standard methods and as described (Delagrave, et al., Protein Engineering, 2001, 14, 261 and U.S. Pre-grant Publication No. 20010051369, each of which is incorporated herein by reference in its entirety). Clone 8-1 differs from K3 by 3 mutations encoding amino acid substitutions C383S, Y436H and V494A. The PCR products were purified by agarose gel electrophoresis and gel extraction using a Qiaquick kit (Qiagen Inc.), resulting in solutions of approximately 200 ng/μL.

Digestion

Three Fok I digests were prepared in 0.2 mL thin-walled PCR tubes (Applied Biosystems, Inc.): Reaction #4′ contained 4 μL of 8-1 PCR DNA and 4 μL of K3 PCR DNA (a total of ˜1.6 μg of DNA). To the DNA were also added 4 μL of 10× buffer M (Roche, Indianapolis, Ind.), 0.8 μL each of oligonucleotide adapter fokGO1392 (5′-CACATCCGTGCACGGATGTGACCCGGTACCTCTCCCC-3′ (SEQ ID NO: 19)), GOFokI 1 (sequence provided in Example 1, above), GOFok12 (see sequence in Example 1), each at a concentration of 25 μM and 25.6 μL of water. Reaction #4− was identical except that the oligonucleotide adapters were replaced with water to provide a negative control reaction. Reaction #8 was prepared identically to reaction #4′, except that only 23.2 μL of water were added.

Each reaction was heated to 95° C. for 1.5 minutes in a 9700 thermocycler (Applied Biosystems Inc.) and cooled as rapidly as the instrument could to 37° C. The enzyme FokI (Roche) was then added to each reaction: reactions #4′ and #4− each received 1.6 μL (6.4 Units) of enzyme while reaction #8 received 4 μL (16 Units). The reactions were then allowed to incubate at 37° C. for 20 minutes, followed by a 20 minute incubation at 65° C. to inactivate the enzyme.

Ligation

Half the volume of each reaction (20 μL) was purified using a Qiaquick kit (Qiagen Inc.) using 35 μL of water to elute the purified, digested DNA. This volume was brought down to 20 μL by use of a vacuum lyophilizer (SpeedVac, Savant Inc.). To each sample, 3 μL of 10× ligation buffer (Fast-Link DNA ligation kit, Epicentre Technologies) were added and the samples were heated to 95° C. for 1.5 minutes and cooled at a rate of 2° C./min, over 35 minutes, to 25° C. Immediately after the samples reached 25° C., 3 μL of 10 mM ATP (provided with Fast-Link kit) and 2 μL of DNA ligase (provided with kit) were added to each sample and the resulting solutions were allowed to incubate 15 minutes at the same temperature.

Gel Purification and PCR Amplification

The ligated DNA was electrophoresed on a 1% agarose gel according to standard methods and bands of 2 kb were excised for each ligation sample. A Qiaquick gel extraction kit (Qiagen) was used to extract the DNA from the gel fragments. The ligated DNA was eluted in a volume of 35 μL of H₂O and 5 μL from each sample were amplified by PCR according to standard methods. (Each PCR contained either 5 μL of ligated DNA from reaction 4′, reaction 4−, reaction 8, no DNA (negative control) or pBADGOK3 (positive control). In addition, each PCR also contained 5 μL of 10× ThermoPol buffer (New England Biolabs), 5 μL 2 mM dNTPs, 1 μL 25 μM Xhosns oligo, 1 μL 25 μM 3′GO oligo, 1 μL Vent polymerase (New England Biolabs), 32 μL H₂O. The resulting mixtures were denatured for 1.5 minutes at 95° C., followed by 25 cycles of denaturation, annealing and extension at 95, 50 and 72° C. for 15, 30 and 105 seconds, respectively. After a further incubation at 72° C. for 5 minutes, the reactions were cooled and stored at 4° C.) Agarose gel electrophoresis of 5 μL aliquots of the PCRs revealed the expected pattern of bands and the remaining 45 μL of the PCRs were purified using a Qiaquick PCR purification kit (Qiagen).

Molecular Cloning of Amplified DNA

The purified amplified DNA was digested with XhoI and HindIII and cloned into vector pBADGOK3 (a derivative of Invitrogen's pBADmyc/hisA) according to standard methods and as described in, e.g., Delagrave, et al., Protein Engineering, 2001, 14, 261 and U.S. Pre-grant Publication No. 20010051369, each of which is incorporated herein by reference in its entirety. Small libraries of clones were thereby generated. In each library, approximately 30% of the clones were actually generated by religation of the vector. Simple optimization of conditions according to standard methods can easily reduce this background to less than 10% of library clones.

Ten transformants from each library (4′, 4− and 8) were picked randomly and sequenced using a 310 Genetic Analyzer (Applied Biosystems) according to methods prescribed by the instrument manufacturers. Results of the sequencing are summarized in the tables below. In Table 1, Clone K3 (WT) has amino acids C, Y and V at positions 383, 436 and 494, respectively, while clone 8-1 has amino acids S, H and A at the same positions. Recombined clones (RECOMB.) are expected to have different combinations of these mutations. Clones 4a to 4j were obtained from reaction #4′, 8a to 8j were from reaction #8 and 4− a to 4− j were from reaction #4−.

The results listed in Table 1 suggest that recombination between parent sequences K3 and 8-1 occurred more efficiently in reactions #4′ and #8, as compared with negative control reaction #4−. Clones 4f, 4g, 4i, 8a and 8f are the products of recombination events. The oligonucleotide adapters used were designed to cause cleavage—and, therefore, recombination—between positions 383 and 436 as well as between positions 436 and 494. Recombination is observed at both sites: clones 4f, 4g, 4i and 8a are due to recombination between residues 383 and 436 while clone 8f is due to recombination between residues 436 and 494. The one recombinant that was found in reaction #4− can be due to a contaminant. Table 2 summarizes and compares the experimental results for each reaction.

Of the ten clones picked from each reaction, about 30% are actually wildtype (WT or K3) background due to the inefficiency of the molecular cloning alluded to above. Therefore, the efficiency of recombination for reaction #4′ is at least 30% and probably closer to 40% while that for reaction #8 is at least 20% and probably closer to 30% (see Table 2). Optimization of conditions is expected to improve the recombination efficiency further, however, the observed recombination frequency is amply sufficient to evolve efficiently genes and/or their proteins.

TABLE 1
Summary of sequencing results.

| | Amino acid at | Amino acid at | Amino acid at | | |
|---|---|---|---|---|---|
| Clone name | position 383 | position 436 | position 494 | Conclusion | Comment |
| 4a | C | Y | V | WT | |
| 4b | C | Y | V | WT | |
| 4c | C | Y | V | WT | |
| 4d | S | H | A | 8-1 | |
| 4e | C | Y | V | WT | |
| 4f | S | Y | V | RECOMB. | S383 is from 8- |
| | | | | | 1 and Y436 |
| | | | | | and V494 are |
| | | | | | from WT |
| 4g | S | Y | V | RECOMB. | S383 is from 8- |
| | | | | | 1 and Y436 |
| | | | | | and V494 are |
| | | | | | from WT |
| 4h | S | H | A | 8-1 | |
| 4i | C | H | A | RECOMB. | C383 is from |
| | | | | | WT and H436 |
| | | | | | and A494 are |
| | | | | | from 8-1 |
| 4j | C | Y | V | WT | |
| 8a | S | Y | V | RECOMB. | S383 is from 8- |
| | | | | | 1 and Y436 |
| | | | | | and V494 are |
| | | | | | from WT |
| 8b | S | H | A | 8-1 | |
| 8c | C | Y | V | WT | |
| 8d | C | Y | V | WT | |
| 8e | C | Y | V | WT | |
| 8f | S | H | V | RECOMB. | S383 and H436 |
| | | | | | are from 8-1 |
| | | | | | and V494 is |
| | | | | | from WT |
| 8g | C | Y | V | WT | |
| 8h | S | H | A | 8-1 | |
| 8i | C | Y | V | WT | |
| 8j | | | | | undetermined |
| 4-a | S | H | A | 8-1 | |
| 4-b | C | Y | V | WT | |
| 4-c | S | H | A | 8-1 | |
| 4-d | C | Y | V | WT | |
| 4-e | C | Y | V | WT | |
| 4-f | C | Y | V | WT | |
| 4-g | C | Y | V | WT | |
| 4-h | S | H | A | 8-1 | |
| 4-i | C | Y | V | WT | |
| 4-j | S | Y | V | RECOMB. | |

TABLE 2
Summary of results listed in Table 1

| | Number of non- | Number of |
|---|---|---|
| | recombined clones | recombined clones |
| Reaction # | found | found |
| 4′ | 7 | 3 |
| 8^{a} | 7 | 2 |
| 4- (negative control) | 9 | 1 |

^{a}One clone undetermined.

For statistical purposes, additional clones from the above experiment were subsequently sequenced and the results listed below in Table 3.

TABLE 3
Summary of subsequent sequencing results

| | Number of non- | Number of |
|---|---|---|
| | recombined clones | recombined clones |
| Reaction # | found | found |
| 4′ | 8 | 2 |
| 8 | 10 | 0 |
| 4- (negative control) | 10 | 0 |

As is apparent from both Tables 2 and 3, a greater number of recombinants were found for 4′ (5 of 20 clones tested) than for controls 8 and 4− (totals of 2 and 1 of 20 clones tested, respectively), as would be expected if the method worked. While the results are not statistically significant based on the chi-squared test, sequencing of yet further clones could lead to statistically improved results, and continued testing and optimization of the method could lead to better recombination efficiencies.

In an experiment similar to above, recombined clones were identified using a galactose oxidase activity assay rather than using sequencing methods. According to this experiment, shuffling was performed by mixing two engineered GO clones (C1 having the mutation C383Stop and F1 having the mutation F453Stop, both engineered into plasmid pBADGOK3 by site-directed mutagenesis) with the adapter oligonucleotides as described above (fokGO1392, GOFok11, and GOFok12). Three reactions were carried out as described above; #1, #2 (no oligonucleotide adapters), and (#3 (no FokI enzyme).) The putatively shuffled DNA samples were cloned according to standard methods and the cloning efficiency for all samples was >90%, with >10⁴ transformants per mL. Resulting transformants were assayed for GO activity according to methods known in the art. All samples had a similar low number of active transformants (2.5 to 3.8%) suggesting that conditions should be improved to make the shuffling experiment more robust.

### Example 5

### Shuffling of Galactose Oxidase (GO) Mutants Using Defined Sequence Adapter Oligonucleotides

Plasmid DNA of 8 GO clones (K3, 8-1, 7.3.2, 7.5.2, GO0.05heat1C, GO0.1heat1C, 8-1heatA and 8-1heat3A; see, e.g., U.S. Pre-grant Publication No. 20010051369, which is incorporated herein by reference in its entirety) was mixed to give a final concentration of about 100 ng/μL and amplified as described in Example 4. The PCR products were purified by agarose gel electrophoresis and gel extraction using a Qiaquick kit (Qiagen Inc.), resulting in solutions of approximately 200 ng/μL.

Four Fok I digests (G1, G2, Ge−, Go−) were prepared in 0.2mL thin-walled PCR tubes (Applied Biosystems, Inc.): Reactions contained 4 μL of each of the 8 different PCR DNAs (about 4 μL each). To the DNA were also added 4 μL of 10× buffer M (Roche, Indianapolis, Ind.), 0.8 μL each of oligonucleotide adapters GOfok10 , GOfok11, GOfok12, GOfok13, GOfok14, and fokGO1392, each at a concentration of 25 μM and 25.6 μL of water. Adapters were omitted from reaction Go− which served as a negative control.

Each reaction was heated to 95° C. for 1.5 minutes in a 9700 thermocycler (Applied Biosystems Inc.) and cooled as rapidly as the instrument could to 37° C. The enzyme FokI (Roche) was then added to each reaction except Ge− (negative control): reactions received 1 μL of enzyme except G2 which received 4 μL (16 Units). The reactions were then allowed to incubate at 37° C. for 20 minutes, followed by a 20 minute incubation at 65° C to inactivate the enzyme.

The DNA was ligated, gel-purified, and amplified as described in Example 4. The resulting PCR products were cloned into pBADGOK3 as previously described. The cloning efficiencies for G1, G2, Go−, and Ge− were >83%, 71%, (63%, and >80%,) respectively. Clones were picked at random and sequenced (Lark Technologies). Results are provided in Table 5 below.

TABLE 5
Summary of sequencing results

| | Number of non- | Number of |
|---|---|---|
| | recombined clones | recombined clones |
| Reaction # | found | found |
| G1 | 9 | 1 |
| G2 | 6 | 2 |
| Ge- (neg. control) | 8 | 0 |
| Go- (neg. control) | 7 | 0 |

As is apparent from Table 5, a greater number of recombinants were found for reactions G1 and G2 than for controls Ge− and Go−, as would be expected if the method worked. While the results are not statistically significant based on the chi-squared test, sequencing of yet further clones could lead to statistically improved results, and continued testing and optimization of the method could lead to better recombination efficiencies.

### Example 6

### Shuffling Using Native Class IIS Restriction Site

According to this experiment, the GO gene (clone K3, wildtype) was amplified by PCR and digested with the enzyme FokI at 37 C for 20 minutes. After heat-inactivation of the enzyme for 20 minutes at 65 C, the digested DNA was purified with a PCR purification kit (Qiagen) and ligated with using a DNA ligation kit (Epicentre). Agarose gel electrophoresis showed that >90% of the digested DNA was ligated to a molecule of the original size (˜2 kb) and that this molecule has the same restriction pattern as an undigested molecule. This result suggests that a gene can be fragmented by digestion with a class IIS restriction enzyme and ligated back (using T4 DNA ligase) to its original size and sequence with high yield.

Applying the above results, each of the five FokI sites present in the GO gene represents a fixed recombination point. To illustrate, a population of eight GO mutants (K3, 8-1, 7.3.2, 7.5.2, GO0.05heat1C, GO0.1heat1C, 8-1heatA and 8-1heat3A; see, e.g., U.S. Pre-grant Publication No. 20010051369, which is incorporated herein by reference in its entirety) were selected and treated as described in Example 5. While oligonucleotide adapters were used, they were unnecessary to carry out the shuffling since naturally occurring FokI restriction sites were present. The shuffled clones were screened. Using methods described in the art (e.g., Delagrave, et al., Protein Engineering, 2001, 14, 261 and U.S. Pre-grant Publication No. 20010051369, each of which is incorporated herein by reference in its entirety), two mutants referred to as G122 and G111 were isolated showing increased activity compared to one of the parent (GO8-1heat3A) on 20 mM methyl-galactose. These clones were sequenced and the observed mutation pattern of G122 clearly shows that recombination occurred incorporating DNA from four of the eight parental clones. The sequence of G122 can be thought of as a composite of four sequence blocks, each from a separate parent and each being flanked by a native FokI restriction site. Data is shown in Table 6 below. Substitutions in G122 are in bold. Substitution I239F in G122 may have arisen during PCR amplification.

TABLE 6
Comparison of G122 mutation pattern with parental clones

| Mutant | Amino acid substitutions | | | | | | | | |
|---|---|---|---|---|---|---|---|---|---|
| G122 | Q63K | G195E | I239F | T352S | K366R | C383S | Y436H | V494A | R636H |
| 7.3.2 | | | K248E | T352S | K366R | C383S | Y436H | V494A | |
| 7.5.2 | | V268E | M278V | S306T | G376S | C383S | Y436H | V494A | R636H |
| GO.05heat1C | | G195E | | | | | | | |
| GO8-1heat3A | Q63K | G195A | | | | | | | |

What is claimed is:

1. A method of preparing a library of polynucleotides comprising:
a) contacting a parent set of polynucleotides with at least one class IIS restriction enzyme to form a plurality of polynucleotide fragments, wherein members of said set of polynucleotides comprise at least one common class IIS restriction site capable of being cleaved by said at least one class IIS restriction enzyme;
b) inactivating said at least one class IIS restriction enzyme or separating said at least one class IIS restriction enzyme from said fragments; and
c) ligating said fragments to yield full-length polynucleotides while allowing for the interchange of analogous fragments, thereby forming said library of polynucleotides.

2. The method of claim 1 wherein said parent set of polynucleotides is at least about 70% homologous.

3. The method of claim 1 wherein said at least one corresponding class IIS restriction enzyme is FokI.

4. The method of claim 1 wherein said members of said parent set of polynucleotides comprise more than one class IIS restriction site.

5. The method of claim 1 wherein said parent set of polynucleotides is contacted with more than one class IIS restriction enzyme.

6. The method of claim 1 wherein said inactivating is carried out by heat inactivation.

7. The method of claim 1 wherein said separating is carried out by purification of said fragments.

8. The method of claim 1 wherein said ligating is carried out with a DNA ligase.

9. The method of claim 8 wherein said DNA ligase is T4 DNA ligase.

10. A library of polynucleotides prepared by the method of claim 1.

11. A method of preparing a polynucleotide with a predetermined property, comprising generating a library of polynucleotides according to the method of claim 1, and identifying at least one polynucleotide within said library having said predetermined property.

12. The method of claim 11 wherein said predetermined property relates to a structural feature, enzymatic activity, or ligand binding affinity.

13. A method of preparing a polynucleotide with a predetermined property comprising:
a) generating a library of polynucleotides according to the method of claim 1;
b) identifying at least one polynucleotide within said library having said predetermined property; and
c) repeating steps a) and b) wherein at least one fragment of said identified polynucleotides is preferentially incorporated into said library.

14. The method of claim 13 wherein said predetermined property relates to a structural feature, enzymatic activity, or ligand binding affinity.

15. A method of preparing a library of polynucleotides comprising:
a) contacting a parent set of polynucleotides with a cleaving enzyme and at least one oligonucleotide adapter, wherein said oligonucleotide adapter directs cleavage of at least two polynucleotides within said set at homologous sites to form a plurality of polynucleotide fragments;
b) ordering said fragments by hybridization with at least one template, allowing for the interchange of analogous fragments, wherein fragment ends resulting from cleavage using a common oligonucleotide adapter are adjacently positioned by said at least one template; and
c) coupling said hybridized fragments to form said library of polynucleotides.

16. The method of claim 15 wherein said parent set of polynucleotides is at least about 70% homologous.

17. The method of claim 15 wherein said parent set of polynucleotides is less than about 70% homologous.

18. The method of claim 15 wherein said cleaving enzyme is a restriction enzyme or nuclease.

19. The method of claim 15 further comprising separating said oligonucleotide adapter and said cleaving enzyme from said fragments prior to said ordering.

20. The method of claim 15 wherein said polynucleotide members are RNA.

21. The method of claim 15 wherein said polynucleotide members are DNA.

22. The method of claim 15 wherein said at least one template is a bridging oligonucleotide.

23. The method of claim 15 wherein said ordering and coupling are repeated until full length polynucleotides are assembled.

24. The method of claim 15 wherein said coupling is carried out with a ligase.

25. The method of claim 24 wherein said ligase is DNA ligase.

26. The method of claim 15 wherein said adapter is defined.

27. The method of claim 15 wherein said adapter is random.

28. A library of polynucleotides prepared by the method of claim 15.

29. A method of preparing a polynucleotide with a predetermined property, comprising generating a library of polynucleotides according to the method of claim 15 , and identifying at least one polynucleotide within said library having said predetermined property.

30. The method of claim 29 wherein said predetermined property relates to a structural feature, enzymatic activity, or ligand binding affinity.

31. A method of preparing a polynucleotide with a predetermined property comprising:
a) generating a library of polynucleotides according to the method of claim 15;
b) identifying at least one polynucleotide within said library having said predetermined property; and
c) repeating steps a) and b) wherein at least one fragment of said identified polynucleotides is preferentially incorporated into said library.

32. The method of claim 31 wherein said predetermined property relates to a structural feature, enzymatic activity, or ligand binding affinity.

33. A method of preparing a library of polynucleotides comprising:
a) contacting a parent set of polynucleotides with a restriction enzyme and at least one oligonucleotide adapter, wherein said adapter comprises a first region capable of hybridizing to at least one region of sequence homologous among said polynucleotide members and a second region comprising a recognition site for said restriction enzyme, wherein cleavage of said polynucleotides at homologous sites among said polynucleotides forms a plurality of polynucleotide fragments;
b) ordering said fragments by hybridization with at least one template, allowing for the interchange of analogous fragments, wherein fragment ends resulting from cleavage using a common oligonucleotide adapter are adjacently positioned by said at least one template; and
c) coupling said hybridized fragments to form said library of polynucleotides.

34. The method of claim 33 wherein said parent set of polynucleotides is at least about 70% homologous.

35. The method of claim 33 wherein said parent set of polynucleotides is less than about 70% homologous.

36. The method of claim 33 wherein said restriction enzyme is a class IIS restriction enzyme.

37. The method of claim 36 wherein said restriction enzyme is FokI.

38. The method of claim 33 further comprising the step of separating said adapter and said restriction enzyme from said fragments prior to said ordering.

39. The method of claim 33 wherein said polynucleotide members are double stranded.

40. The method of claim 39 wherein said fragments also serve as templates for said ordering.

41. The method of claim 33 wherein said at least one template is a bridging oligonucleotide.

42. The method of claim 33 wherein said ordering and coupling are repeated until full length polynucleotides are assembled.

43. The method of claim 33 wherein said coupling is carried out with a ligase.

44. The method of claim 43 wherein said ligase is DNA ligase.

45. The method of claim 33 wherein said adapter is defined.

46. The method of claim 33 wherein said adapter is random.

47. A library of polynucleotides prepared by the method of claim 33.

48. A method of preparing a polynucleotide with a predetermined property, comprising generating a library of polynucleotides according to the method of claim 33, and identifying at least one polynucleotide within said library having said predetermined property.

49. The method of claim 48 wherein said predetermined property relates to a structural feature, enzymatic activity, or ligand binding affinity.

50. A method of preparing a polynucleotide with a predetermined property comprising:
a) generating a library of polynucleotides according to the method of claim 33;
b) identifying at least one polynucleotide within said library having said predetermined property; and
c) repeating steps a) and b) wherein at least one fragment of said identified polynucleotides is preferentially incorporated into said library.

51. The method of claim 50 wherein said predetermined property relates to a structural feature, enzymatic activity, or ligand binding affinity.

52. A method of preparing a library of polynucleotides comprising:
a) contacting a parent set of RNA polynucleotides with a ribonuclease and at least one DNA oligonucleotide adapter to allow cleavage of said RNA polynucleotides at homologous sites, forming a plurality of RNA polynucleotide fragments;
b) ordering said fragments by hybridization with at least one template, allowing for the interchange of analogous fragments, wherein fragment ends resulting from cleavage using a common oligonucleotide adapter are adjacently positioned by said at least one template; and
c) coupling said hybridized fragments to form said library of polynucleotides.

53. The method of claim 52 wherein said parent set of RNA polynucleotides is at least about 70% homologous.

54. The method of claim 52 wherein said parent set of RNA polynucleotides is less than about 70% homologous.

55. The method of claim 52 wherein said ribonuclease is RNase H.

56. The method of claim 52 further comprising the step of separating said adapter and said nuclease from said fragments prior to said ordering.

57. The method of claim 52 wherein said nuclease is inactivated by heating prior to said ordering.

58. The method of claim 52 wherein said at least one template is a bridging oligonucleotide.

59. The method of claim 52 wherein said ordering and coupling are repeated until full length RNA polynucleotides are assembled.

60. The method of claim 52 wherein said coupling is carried out with a ligase.

61. The method of claim 60 wherein said ligase is DNA ligase.

62. The method of claim 52 wherein said adapter is defined.

63. The method of claim 52 wherein said adapter is random.

64. A library of polynucleotides prepared by the method of claim 52.

65. A method of preparing a polynucleotide with a predetermined property, comprising generating a library of polynucleotides according to the method of claim 52, and identifying at least one polynucleotide within said library having said predetermined property.

66. The method of claim 65 wherein said predetermined property relates to a structural feature, enzymatic activity, or ligand binding affinity.

67. A method of preparing a polynucleotide with a predetermined property comprising:
a) generating a library of polynucleotides according to the method of claim 52;
b) identifying at least one polynucleotide within said library having said predetermined property; and
c) repeating steps a) and b) wherein at least one fragment of said identified polynucleotides is preferentially incorporated into said library.

68. The method of claim 67 wherein said predetermined property relates to a structural feature, enzymatic activity, or ligand binding affinity.


SEQUENCE LISTING (19 sequences)

SEQ ID NO: 1 - DNA, 38 bp, Artificial Sequence
  Oligonucleotide adapter
cacatccgtgcacggatgtgtcctgagccttcgagcct

SEQ ID NO: 2 - DNA, 33 bp, Artificial Sequence
  Oligonucleotide adapter
gtgtgccaaaaggtatcacatccgtaggatgtg

SEQ ID NO: 3 - DNA, 33 bp, Artificial Sequence
  Oligonucleotide adapter
gcagggcgagtttcaacacatccgtaggatgtg

SEQ ID NO: 4 - DNA, 37 bp, Artificial Sequence
  Oligonucleotide adapter
cacatccgtgcacggatgtgctggtcttggacgctgg

SEQ ID NO: 5 - DNA, 34 bp, Artificial Sequence
  Oligonucleotide adapter
gatcccctggtggtatccacatccgtaggatgtg

SEQ ID NO: 6 - DNA, 33 bp, Artificial Sequence
  Oligonucleotide adapter
ccgtctgacatggtagcacatccgtaggatgtg

SEQ ID NO: 7 - DNA, 37 bp, Artificial Sequence
  Oligonucleotide adapter
cacatccgtgcacggatgtgaggtcaacccaatgttg

SEQ ID NO: 8 - DNA, 33 bp, Artificial Sequence
  Oligonucleotide adapter
ccactggtatagtacccacatccgtaggatgtg

SEQ ID NO: 9 - DNA, 36 bp, Artificial Sequence
  Oligonucleotide adapter
cacatccgtgcacggatgtgtcctgacctttggcgg

SEQ ID NO: 10 - DNA, 35 bp, Artificial Sequence
  Oligonucleotide adapter
gaaacgttcgggcaaagtcacatccgtaggatgtg

SEQ ID NO: 11 - DNA, 37 bp, Artificial Sequence
  Oligonucleotide adapter
cacatccgtgcacggatgtgacgtccctgaacaagac

SEQ ID NO: 12 - DNA, 35 bp, Artificial Sequence
  Oligonucleotide adapter
gattcgtggtacaatcgccacatccgtaggatgtg

SEQ ID NO: 13 - DNA, 36 bp, Artificial Sequence
  Oligonucleotide adapter
cacatccgtgcacggatgtgtcggcggccgcattac

SEQ ID NO: 14 - DNA, 34 bp, Artificial Sequence
  Oligonucleotide adapter
gctatttcctccattgtcacatccgtaggatgtg

SEQ ID NO: 15 - DNA, 20 bp, Artificial Sequence
  primer oligonucleotide
ctactgtttctccatacccg

SEQ ID NO: 16 - DNA, 20 bp, Artificial Sequence
  primer oligonucleotide
aaacagccaagctggagacc

SEQ ID NO: 17 - DNA, 26 bp, Artificial Sequence
  Oligonucleotide adapter
cacatccgtgcacggatgtgnnnnnn

SEQ ID NO: 18 - DNA, 26 bp, Artificial Sequence
  Oligonucleotide adapter
cacatccgtgcacggatgtgatgnnn

SEQ ID NO: 19 - DNA, 37 bp, Artificial Sequence
  Oligonucleotide adapter
cacatccgtgcacggatgtgacccggtacctctcccc